(19) United States
(12) Patent Application Publication
(10) Pub. No.: US 2026/0027332 A1
(43) Pub. Date: Jan. 29, 2026

(54) GUIDEWIRE AND MEDICAL DEVICE INCLUDING LASER CUT TUBE HAVING MULTIPLE CONNECTOR BEAM GEOMETRY

(71) Applicant: Deepin Technologies, LLC, San Jose, CA (US)

(72) Inventors: Alex Kubacki, San Jose, CA (US); Jiaxu Wang Wang, San Jose, CA (US)

(73) Assignee: Deepin Technologies, LLC, San Jose, CA (US)

(21) Appl. No.: 19/251,687

(22) Filed: Jun. 26, 2025

Related U.S. Application Data
(60) Provisional application No. 63/675,598, filed on Jul. 25, 2024.

(51) Int. Cl. A61M 25/09; A61M 25/00

(52) U.S. Cl. CPC A61M 25/09; A61M 25/0013; A61M 2025/09083; A61M 2025/09108

(57) ABSTRACT

A guidewire device includes a core wire and a tube member located near the distal end of the core wire. The tube member comprises a segment provided with a plurality of transverse cuts at a plurality of axial locations along a longitudinal axis of the tube member. The plurality of transverse cuts define a plurality of rings extending circumferentially around the longitudinal axis and a plurality of sets of beams extending axially along the longitudinal axis to connect the plurality of rings. Each of the plurality of sets of beams comprises a first beam and a second beam spaced apart from each other at a radial angle of non-180 degrees. The radial angles of the plurality of sets of beams change successively along the longitudinal axis of the tube member.

### CROSS-REFERENCE TO RELATED APPLICATIONS

This application claims priority to U.S. provisional patent application No. 63/675,598 filed Jul. 25, 2024 entitled “Multiple Connector Beam Geometry for Guide Wires,” the disclosure of which is hereby incorporated by reference in its entirety.

### TECHNICAL FIELD

This application relates generally to medical devices and methods of making and using medical devices to treat diseases. Particularly, various embodiments of guidewire devices and methods are described.

### BACKGROUND

Guidewire devices are widely used in the medical field for guiding an ancillary device to a particular location in a patient's body to perform delicate procedures e.g., guiding a catheter deep into the vasculature of the body. Guidewire devices often require a variable stiffness profile, typically with the most flexible section at the distal end while maintaining good torque transmission for trackability and delivery in tortuous anatomy.

A guidewire device generally includes a core wire, which may have a tapered distal section reinforced with a structure joined to an atraumatic tip. Traditionally, metal coils or braids are used as guidewire reinforcement. As micro-machining and laser cutting technologies have evolved, slotted hypotubes have also entered the field as device components.

While advancement has been made in the field of guidewire devices, there is still a general need for improvement. It would be desirable to provide improved guidewire devices that can balance the complex bending flexibility, tensile strength, and torque transmission requirements for various medical applications.

### SUMMARY

In one aspect, embodiments of the disclosure feature a guidewire device. In general, an embodiment of the guidewire device comprises a core wire extending between a proximal end and a distal end and a tube member located near the distal end of the core wire. The tube member comprises a segment provided with a plurality of transverse cuts at a plurality of axial locations along a longitudinal axis of the tube member. The plurality of transverse cuts define a plurality of rings extending circumferentially around the longitudinal axis and a plurality of sets of beams extending axially along the longitudinal axis to connect the plurality of rings. Each of the plurality of sets of beams comprises a first beam and a second beam spaced apart from each other at a radial angle of non-180 degrees. The radial angles of the plurality of sets of beams change successively along the longitudinal axis of the tube member.

In another aspect, embodiments of the disclosure feature a tube member for use in a medical device. In general, an embodiment of the tube member comprises a segment provided with a plurality of transverse cuts at a plurality of axial locations along a longitudinal axis of the tube member. The plurality of transverse cuts define a plurality of rings extending circumferentially around the longitudinal axis and a plurality of sets of beams extending axially along the longitudinal axis to connect the plurality of rings. Each of the plurality of sets of beams comprises a first beam and a second beam spaced apart from each other at a radial angle of non-180 degrees. The radial angles of the plurality of sets of beams change successively along the longitudinal axis of the tube member.

This Summary is provided to introduce selected aspects and embodiments of this disclosure in a simplified form and is not intended to identify key features or essential characteristics of the claimed subject matter, nor is it intended to be used as an aid in determining the scope of the claimed subject matter. The selected aspects and embodiments are presented merely to provide the reader with a summary of certain forms the invention might take and are not intended to limit the scope of the invention. Other aspects and embodiments of the disclosure are described in the section of Detailed Description.

These and various other aspects, embodiments, features, and advantages of the disclosure will become better understood upon reading of the following detailed description in conjunction with the accompanying drawings.

### BRIEF DESCRIPTION OF THE DRAWINGS

FIG. 1 is a simplified illustration of an example guidewire device according to embodiments of the disclosure.

FIG. 2 is a simplified illustration of the guidewire device of FIG. 1 with components separated to show the core wire, the tube member, and other components with greater clarity.

FIG. 3 is a simplified illustration of a segment of an example tube member according to embodiments of the disclosure.

FIG. 4A is a cross-sectional view of the segment of the tube member shown in FIG. 3, taken along lines 4-4.

FIG. 4B is a cross-sectional view of the segment of the tube member shown in FIG. 3, taken along lines 4′-4′.

FIG. 5 depicts an example tube member including a plurality of cuts according to embodiments of the disclosure.

FIG. 6 shows cross-sectional views of a distal portion of the example tube member of FIG. 5 at a plurality of axial locations of the tube member.

FIG. 7 shows cross-sectional views of a first segment of a mid-portion of the example tube member of FIG. 5 at a plurality of axial locations of the tube member.

FIG. 8 shows cross-sectional views of a second segment of a mid-portion of the example tube member of FIG. 5 at a plurality of axial locations of the tube member.

FIG. 9 shows a cross-sectional view of an example tube member according to embodiments of the disclosure.

### DETAILED DESCRIPTION OF EMBODIMENTS

With reference to the figures, various embodiments of guidewire devices and methods will now be described. The figures are intended to facilitate description of embodiments of the disclosure and are not necessarily drawn to scale. Certain specific details may be set forth in the figures to provide a thorough understanding of the disclosure. It will be apparent to one of ordinary skill in the art that some of these specific details may not be employed to practice embodiments of the disclosure. In other instances, structures, components, systems, materials, and/or operations often associated with known medical procedures may not be shown or described in detail to avoid unnecessarily obscuring description of embodiments of the disclosure.

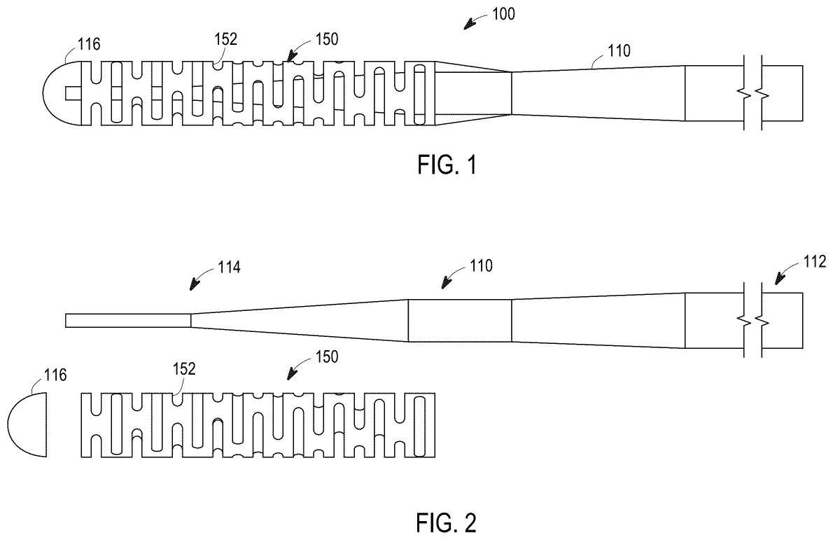

FIGS. 1-2 schematically illustrate an example guidewire device 100 according to embodiments of the disclosure. The guidewire device 100 is generally configured for use in conjunction with a medical device to perform procedures such as neuro-, cardio-, or peripheral vasculature interventions. One example application of the guidewire device 100 of the disclosure is for guiding a catheter deep within the neuro vasculature. In a broad overview, the guidewire device 100 includes an elongate core wire 110, a tube member 150 coupled to the core wire 110, and an atraumatic tip 116 at the distal end of the guidewire device 100. The core wire 110 extends between a proximal portion 112 and a distal portion 114, and has a length suitable for a particular application. The distal portion 114 of the core wire 110 may be tapered towards the distal end to provide more bending flexibility. The proximal portion 112 of the core wire 110 may have an increased diameter to maintain pushability and torsional rigidity of the guidewire device 100. The tube member 150 may be located near the distal portion 114 of the core wire 110 and secured to the core wire 110 to provide reinforcement and improve performance of the guidewire device 100. The tube member 150 can be secured to the distal portion 114 of the core wire 110 via various means e.g., bonding, welding, soldering, etc. to allow transmission of torsional force from the proximal section 112 of the core wire 110 to the tube member 150 and/or from the tube member 150 to the distal section 114 of the core wire 110. In the space defined between the tube member 150 and the distal portion 114 of the core wire 110, various components such as a radiopaque marker, a centering device, a core wire stiffness indicator etc. (not shown in FIGS. 1-2) can be provided to improve the performance of the guidewire device 100. The tube member 150 can be a hypotube constructed from a shape-memory material, and may include a plurality of cuts 152 configured to improve the effectiveness of the guidewire device 100, e.g., providing a desirable balance between bending flexibility, torsional rigidity, tensile strength, etc. The plurality of cuts 152 may be vertical cuts and/or helical cuts circumferentially extending around the central longitudinal axis of the tube member. U.S. Ser. No. 18/963,683 filed Nov. 28, 2024 entitled “Guidewire and Medical Device including Laser Cut Tube” and U.S. Ser. No. 19/043,429 filed Feb. 1, 2025 entitled “Intravascular Medical Devices Including Laser Cut Tube” describe various embodiments of cut-tube structures which can be used as the tube member 150 of the guidewire device 100. The disclosures of U.S. Ser. Nos. 18/963,683 and 19/043,429 are hereby incorporated by reference in their entirety.

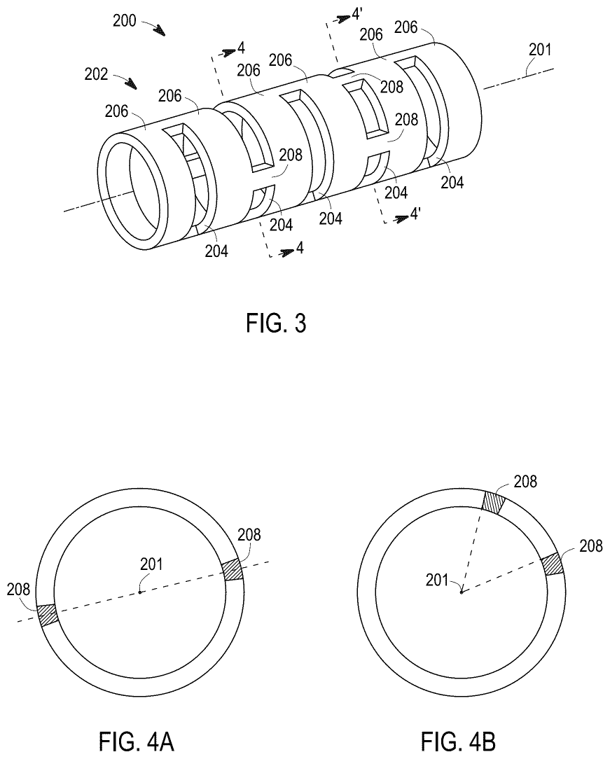

FIG. 3 depicts an example tube member 200 which can be used as a component 150 of the guidewire device 100 according to embodiments of the disclosure. As shown, the example tube member 200 comprises a segment 202 which is provided with a plurality of transverse cuts 204 at a plurality of axial locations along a longitudinal axis 201 of the tube member 200. The plurality of transverse cuts 204 define a plurality of rings 206 extending circumferentially around the longitudinal axis 201 and a plurality of sets of beams 208 extending axially along the longitudinal axis 201 to connect the plurality of rings 206.

To facilitate description of embodiments of the disclosure, various terms are used in the Specification and appended Claims. The term “axial location” is used to refer to a location along the longitudinal axis 201 of the tube member 200.

The term “transverse cut” is used herein to refer to a cut or slot in the tube member 200 that extends in a plane transverse to the longitudinal axis 201 of the tube member 200. A transverse cut can be a vertical cut, e.g., formed in a plane generally normal to the longitudinal axis 201 of the tube member 200. A transverse cut can also be a helical cut, e.g., formed at an angle e.g., 5-45 degrees relative to a plane normal to the longitudinal axis 201 of the tube member 200.

The term “ring” is used herein to refer to an uncut, ring-shaped structure in the tube member 200 that extends circumferentially around the longitudinal axis 201 of the tube member 200. The term “ring” may be used interchangeably with “circumferentially extending ring.”

The term “beam” is used herein to refer to an uncut section in the tube member 200 that connects adjacent circumferentially extending rings. The term “beam” may be used interchangeably with the term “axially extending beam” since a beam extends along the longitudinal axis 201 of the tube member 200 in connecting adjacent circumferentially extending rings. According to embodiments of the disclosure, a set of two or more beams connect two adjacent rings. For example, in a two-beam connector geometry as shown in FIG. 3, two axially extending beams forms a set to connect adjacent circumferentially extending rings. The set of beams can be arranged symmetrically, for example, two beams of a set are circumferentially equally spaced apart (e.g., opposite to each other in 180 degrees) as shown in FIG. 4A. Alternatively, the set of beams can be arranged asymmetrically, for example, two beams of a set are brought closer together circumferentially (less than 180 degrees) or spaced apart farther (greater than 180 degrees), as shown in FIG. 4B. The term “radial angle” refers to the angular degree spanning between the two beams in a set with respect to the central axis 201 of the tube member 200, and can be used to indicate the distance between the two beams spaced apart in the set. The “radial angle” can be defined as the angle formed between the lines extended from the middles of the two beams of the set as shown in FIG. 4A, or between the lines extended from the lateral surfaces of the two beams of the set as shown in FIG. 4B.

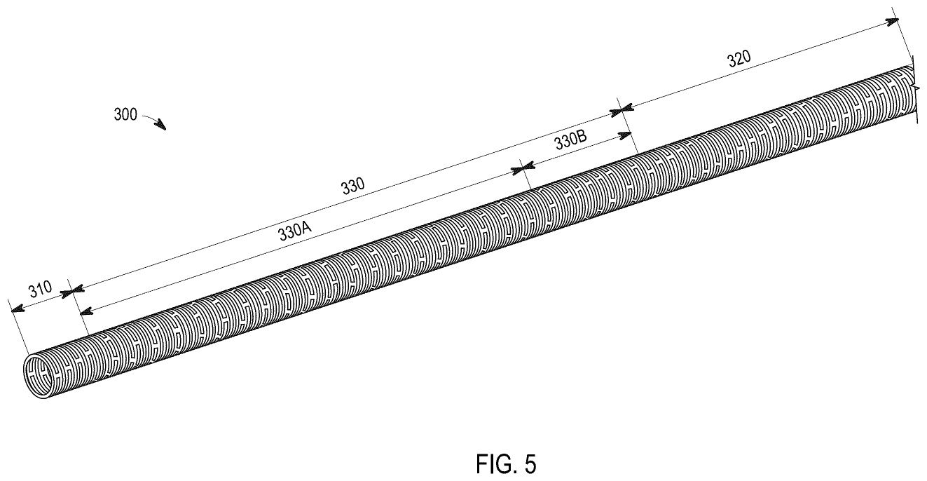

With reference now to FIG. 5, an example tube member 300 according to embodiments of the disclosure comprises a distal portion 310, a proximal portion 320, and a mid-portion 330 between the distal portion 310 and the proximal portion 320. The mid-portion 330 may include two or more segments such as a first segment 330A and a second segment 330B as shown. According to embodiments of the disclosure, each of the distal portion 310, proximal portion 320, and the mid-portion 330 of the tube member 300 is provided with a plurality of transverse cuts, with geometries and/or features configured to balance the complex bending flexibility, tensile strength, and torque transmission requirements for various medical applications.

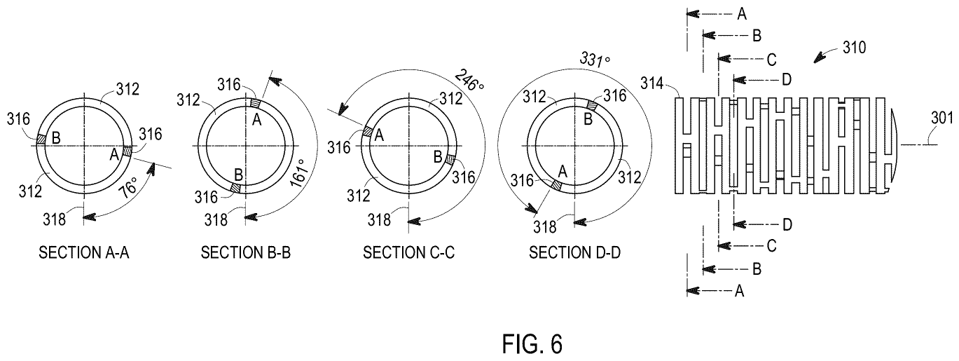

FIG. 6 shows cross-sectional views (left panels) of the distal portion 310 of the example tube member 300 taken at different axial locations, and a side view (right panel) of the distal portion 310. As shown in FIG. 6, the distal portion 310 of the tube member 300 is provided with a plurality of transverse cuts 312 at a plurality of axial locations along the longitudinal axis 301 of the tube member 300. The plurality of transverse cuts 312 define a plurality of rings 314 extending circumferentially around the longitudinal axis 301 and a plurality of sets of beams 316 extending axially along the longitudinal axis 301 to connect the plurality of rings 314. Each of the plurality of sets of beams 316 comprises two beams (A and B in the cross-sectional views), symmetrically spaced apart or opposite to each other in 180 degrees.

With reference to FIG. 6, the successive sets of beams 316 can be rotationally offset from each other along the longitudinal axis 301 of the tube member 300. In the example shown in FIG. 6, the successive sets of beams 316 are rotationally offset with a constant angle of 95 degrees (counterclockwise). For example, from Section A-A where a set of beams 316 align at 76 degrees with respect to a vertical reference 318 to Section B-B where a set of beams 316 align at 161 degrees with respect to the vertical reference 318, the two adjacent sets of beams 316 are rotationally offset by 95 degrees (the difference between 161 degrees and 76 degrees). From Section B-B where a set of beams 316 align at 161 degrees with respect to the vertical reference 318 to Section C-C where a set of beams 316 align at 246 degrees with respect to the vertical reference 318, the two adjacent sets of beams 316 are rotationally offset by 95 degrees (the difference between 246 degrees and 161 degrees). From Section C-C where a set of beams 316 align at 246 degrees with respect to the vertical reference 318 to Section D-D where a set of beams align at 331 degrees with respect to the vertical reference 318, the two adjacent sets of beams 316 are rotationally offset by 95 degrees (the difference between 331 degrees and 246 degrees). In general, the successive sets of beams 316 can be rotationally offset in a linear pattern or at a constant angle ranging from 5 to 175 degrees (counterclockwise or clockwise). In alternative embodiments of the disclosure, the successive sets of beams are rotationally offset in a non-linear pattern or rotated by non-constant angles. In some embodiments, the successive sets of beams can be arranged to align with each other, or to not be rotationally offset.

According to embodiments of the disclosure, the proximal portion 320 of the example tube member 300 can be provided with a plurality of transverse cuts having a cut pattern same as or similar to the cut pattern of the distal portion 310 of the tube member 300 as described above in conjunction with FIG. 6. For example, the proximal portion 320 of the example tube member 300 may be provided with a plurality of transverse cuts at a plurality of axial locations along the longitudinal axis 301 of the tube member 300. The plurality of transverse cuts 312 in the proximal portion 320 define a plurality of rings extending circumferentially around the longitudinal axis 301 and a plurality of sets of beams extending axially along the longitudinal axis 301 to connect the plurality of rings. The successive sets of beams may be rotationally offset with a constant angle e.g., from 5 to 175 degrees (counterclockwise or clockwise). Alternatively, the successive sets of beams of the proximal portion 320 of the tube member 300 are rotationally offset in a non-linear pattern or rotated by non-constant angles. In some embodiments, the successive sets of beams of the proximal portion 320 of the tube member 300 can be arranged to align with each other, or to not be rotationally offset.

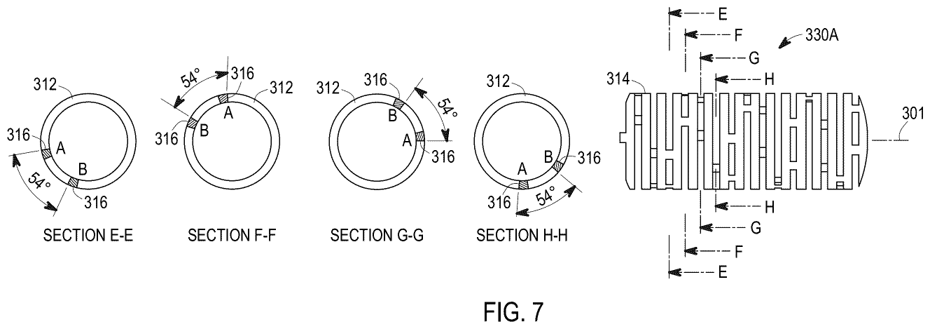

FIG. 7 shows cross-sectional views (left panels) of a segment 330A of the mid-portion 330 of the example tube member 300 taken at different axial locations, and a side view (right panel) of the segment 330A of the mid-portion 330. Similar to the distal portion 310 of the tube member 300, the segment 330A of the mid-portion 330 of the tube member 300 shown in FIG. 7 is provided with a plurality of transverse cuts 312 at a plurality of axial locations along the longitudinal axis 301 of the tube member 300. The plurality of transverse cuts 312 define a plurality of rings 314 extending circumferentially around the longitudinal axis 301 and a plurality of sets of beams 316 extending axially along the longitudinal axis 301 to connect the plurality of rings 314. Each of the plurality of sets of beams 316 comprises two beams (A and B in the cross-sectional views). Different from the distal portion 310 of the tube member 300 shown in FIG. 6, the beams 316 of a set in the segment 330A of the mid-portion 300 shown in FIG. 7 are arranged asymmetrically. In the example shown in FIG. 7, the radial angle between the two beams of a set is 54 degrees. In general, the two beams of a set in the segment 330A of the mid-portion 330 can be spaced apart at any non-180 degrees, bringing the two beams 316 closer together (less than 180 degrees) in one direction or spaced apart farther (greater than 180 degrees) in the other direction. The asymmetrical beam spacing leaves a larger unsupported region, allowing the segment 330A to bend more freely.

With reference to FIG. 7, the successive sets of beams 316 in the segment 330A can be rotationally offset by a constant angle e.g., 95 degrees (clockwise) as shown. For example, from Section E-E to Section F-F, the two adjacent sets of beams 316 are rotationally offset by 95 degrees (clockwise). From Section F-F to Section G-G, the two adjacent sets of beams 316 are rotationally offset by 95 degrees. From Section G-G to Section H-H, the two adjacent sets of beams 316 are rotationally offset by 95 degrees. In general, the successive sets of beams can be rotationally offset in a linear pattern or at a constant angle ranging from 5 to 355 degrees (clockwise or counterclockwise). In alternative embodiments of the disclosure, the successive sets of beams are rotationally offset in a non-linear pattern or rotated by non-constant angles. In some embodiments, the successive sets of beams are not rotationally offset.

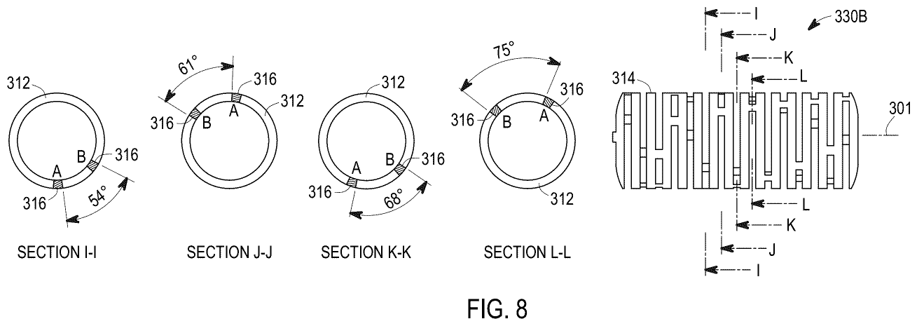

FIG. 8 shows cross-sectional views (left panels) of another segment 330B of the mid-portion 330 of the example tube member 300 taken at different axial locations and a side view (right panel) of the segment 330B of the mid-portion 330. Similar to the segment 330A of the mid-portion 330 of the tube member 300, the segment 330B shown in FIG. 8 is provided with a plurality of transverse cuts 312 at a plurality of axial locations along the longitudinal axis 301 of the tube member 300. The plurality of transverse cuts 312 define a plurality of rings 314 extending circumferentially around the longitudinal axis 301 and a plurality of sets of beams 316 extending axially along the longitudinal axis 301 to connect the plurality of rings 314. Each of the plurality of sets of beams 316 comprises two beams (A and B in the cross-sectional views). The beams 316 of a set are arranged asymmetrically, i.e., the two beams 316 of a set are spaced apart at non-180 degrees, bringing the two beams 316 in a set closer together (less than 180 degrees) in one direction or spaced apart farther (greater than 180 degrees) in the other direction. Different from the segment 330A of the mid-portion 330, the beam radial angles of successive sets of beams in the segment 330B change.

With reference to FIG. 8, according to embodiments of the disclosure, the radial angles of successive sets of beams change at a constant degree. For example, as shown in FIG. 8, the radial angles of successive sets of beams change by 7 degrees. Specifically, from Section I-I to Section J-J, the radial angles increase by 7 degrees (from 54 degrees to 61 degrees). From Section J-J to Section K-K, the radial angles increase by 7 degrees (from 61 degrees to 68 degrees). From Section K-K to Section L-L, the radial angles increase by 7 degrees (from 68 degrees to 75 degrees). In general, the radial angles of successive sets of beams can change at a constant degree ranging from 2 to 25 degrees. By moving one of the beams radially further away or closer to the other gradually, a smooth transition of bending flexibility can be provided. In alternative embodiments of the disclosure, the radial angles of successive sets of beams change at a non-constant degree.

With reference to FIG. 8, the successive sets of beams can be further rotationally offset by a constant angle e.g., 190 degrees as shown (clockwise). For example, from Section I-I to Section J-J, the two sets of beams are rotationally offset by 190 degrees (clockwise). From Section J-J to Section K-K, the two sets of beams are rotationally offset by 190 degrees (clockwise). From Section K-K to Section L-L, the two sets of beams are rotationally offset by 190 degrees (clockwise). In general, the successive sets of beams can be rotationally offset in a linear pattern or at a constant angle ranging from 5 to 355 degrees (clockwise or counterclockwise). Alternatively, the successive sets of beams can be rotationally offset in a non-linear pattern or at a non-constant angle.

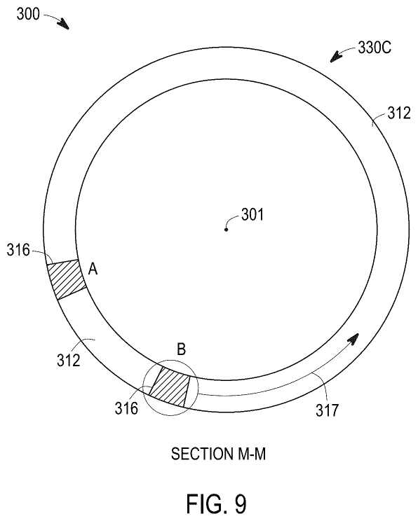

FIG. 9 shows a cross-sectional view of an example tube member 300 according to alternative embodiments of the disclosure. According to the alternative embodiments of the disclosure, a segment 330C of the tube member 300 is provided with a plurality of transverse cuts 312 at a plurality of axial locations along the longitudinal axis 301 of the tube member 300. The plurality of transverse cuts 312 define a plurality of rings (not shown in FIG. 9) extending circumferentially around the longitudinal axis 301 and a plurality of sets of beams 316 extending axially along the longitudinal axis 301 to connect the plurality of rings. Each of the plurality of sets of beams 316 comprises two beams (A and B in the cross-sectional view). The beams 316 of a set in the segment 330C of the tube member 300 are arranged asymmetrically, i.e., the two beams 316 of a set in the segment 330C are spaced apart at non-180 degrees, bringing the two beams closer together (less than 180 degrees) in one direction or spaced apart farther (greater than 180 degrees) in the other direction.

With reference to FIG. 9, one of the two beams 316 in each of successive sets of beams 316 of the segment 330C can be positioned at a fixed or same spatial angle with respect to the central longitudinal axis 301. For example, the beam A in each of the successive sets of beams 316 can be positioned at a fixed spatial angle with respect to the central axis 301. The other beam B in the successive sets of beams 316 changes e.g., increases the radial distance from its paired beam A successively, as indicated by the arrow 317 in FIG. 9. The successive change or increase of the radial angles can be at a constant degree ranging from 2 to 25 degrees. Alternatively, the successive change or increase of the radial angles can be non-linear. By moving one of the beams radially further away or closer to the other gradually, a smooth transition of bending flexibility can be achieved.

Various embodiments of guidewire devices and methods have been described with reference to figures. It should be noted that an aspect described in conjunction with a particular embodiment is not necessarily limited to that embodiment and can be practiced in any other embodiments. The figures are intended for illustration of embodiments but not for exhaustive description or limitation on the scope of the disclosure. Alternative structures, components, and materials will be readily recognized as being viable without departing from the principle of the claimed invention. While some embodiments of the disclosure are described in conjunction with a guidewire device, this is not intended to be limiting. For example, the tube members described herein can be used as a component for other interventional devices such as catheters. Further, while embodiments of a tube member are described in conjunction with a two-beam cut pattern, the principle described herein also applies to other multiple beam connector geometry such as three-beam cut pattern, four-beam cut pattern, and so on.

All technical and scientific terms used herein have the meaning as commonly understood by one of ordinary skill in the art unless specifically defined otherwise. As used in the description and appended claims, the singular forms of “a,” “an,” and “the” include plural references unless the context clearly dictates otherwise. The term “or” refers to a nonexclusive “or” unless the context clearly dictates otherwise. The term “proximal” and its grammatical equivalents refer to a position, direction or orientation towards the user or physician's side. The term “distal” and its grammatical equivalents refer to a position, direction, or orientation away from the user or physician's side. The designations “rearward,” “forward,” and the like are not meant to limit the referenced component to a specific orientation. It will be appreciated that such designations refer to the orientation of the referenced component as illustrated in the Figures; the systems and devices of the disclosure can be used in any orientation suitable to the user. The term “first” or “second” etc. may be used to distinguish one element from another in describing various similar elements. It should be noted the terms “first” and “second” as used herein include references to two or more than two. Further, the use of the term “first” or “second” should not be construed as in any particular order unless the context clearly dictates otherwise. The order in which the method steps are performed may be changed in alternative embodiments. One or more method steps may be skipped altogether, and one or more optional steps may be included. All numeric values are provided for illustration and assumed to be modified by the term “about,” whether explicitly indicated or not. The term “about” generally refers to a range of numbers that one of skill in the art would consider equivalent to the recited value e.g., having the same function or result. The term “about” may include numbers that are rounded to the nearest significant figure. The recitation of a numerical range by endpoints includes all numbers within that range.

Those skilled in the art will appreciate that various other modifications may be made. All these or other variations and modifications are contemplated by the inventors and within the scope of the invention.

What is claimed is:

1. A guidewire device comprising:
a core wire extending between a proximal end and a distal end; and
a tube member located near the distal end of the core wire,
wherein the tube member comprises a segment provided with a plurality of transverse cuts at a plurality of axial locations along a longitudinal axis of the tube member, the plurality of transverse cuts defining a plurality of rings extending circumferentially around the longitudinal axis and a plurality of sets of beams extending axially along the longitudinal axis to connect the plurality of rings, wherein each of the plurality of sets of beams comprises a first beam and a second beam spaced apart from each other at a radial angle of non-180 degrees, and the radial angles of the plurality of sets of beams change successively along the longitudinal axis of the tube member.

2. The guidewire device of claim 1, wherein the radial angles of the plurality of sets of beams change successively at a constant degree.

3. The guidewire device of claim 2, wherein the radial angles of the plurality of sets of beams change successively at a constant degree between 2-25 degrees.

4. The guidewire device of claim 1, wherein the radial angles of the plurality of sets of beams change successively at a non-constant degree.

5. The guidewire device of claim 1, wherein the first beams of the plurality of sets of beams are located at a substantially same spatial angle with respect to the longitudinal axis of the tube member.

6. The guidewire device of claim 5, wherein the radial angles of the plurality of sets of beams change successively at a constant degree between 2-25 degrees.

7. The guidewire device of claim 1, wherein the first beams of the plurality of sets of beams are angularly offset successively along the longitudinal axis of the tube member, and the second beams of the plurality of sets of beams are angularly offset successively along the longitudinal axis of the tube member.

8. The guidewire device of claim 7, wherein the first beams of the plurality of sets of beams are angularly offset successively at a constant degree, and the second beams of the plurality of sets of beams are angularly offset successively at a degree equal to the constant degree plus a constant value.

9. The guidewire device of claim 1, wherein each of the plurality of sets of beams comprises more than two beams.

10. The guidewire device of claim 1, wherein the tube member further comprises a distal portion and a proximal portion positioning the segment of the tube member therebetween, wherein at least one of the distal portion and the proximal portion is provided with a plurality of transverse cuts at a plurality of axial locations along the longitudinal axis of the tube member, the plurality of transverse cuts defining a plurality of rings extending circumferentially around the longitudinal axis and a plurality of sets of beams extending axially along the longitudinal axis to connect the plurality of rings, wherein each of the plurality of sets of beams in the at least one of the distal portion and the proximal portion comprises a first beam and a second beam spaced apart from each other at radial angle of 180 degrees.

11. The guidewire device of claim 10, wherein the plurality of sets of beams are rotationally offset successively along the longitudinal axis of the tube member.

12. A tube member for use in a medical device, wherein:
the tube member comprises a segment provided with a plurality of transverse cuts at a plurality of axial locations along a longitudinal axis of the tube member, the plurality of transverse cuts defining a plurality of rings extending circumferentially around the longitudinal axis and a plurality of sets of beams extending axially along the longitudinal axis to connect the plurality of rings, wherein each of the plurality of sets of beams comprises a first beam and a second beam spaced apart from each other at a radial angle of non-180 degrees, and the radial angles of the plurality of sets of beams change successively along the longitudinal axis of the tube member.

13. The tube member of claim 12, wherein the radial angles of the plurality of sets of beams change successively at a constant degree.

14. The tube member of claim 13, wherein the radial angles of the plurality of sets of beams change successively at a constant degree between 2-25 degrees.

15. The tube member of claim 12, wherein the radial angles of the plurality of sets of beams changes successively at a non-constant degree.

16. The tube member of claim 12, wherein the first beams of the plurality of sets of beams are located at a substantially same spatial angle with respect to the longitudinal axis of the tube member.

17. The tube member of claim 16, wherein the radial angles of the plurality of sets of beams change successively at a constant degree between 2-25 degrees.

18. The tube member of claim 12, wherein the first beams of the plurality of sets of beams are angularly offset successively along the longitudinal axis of the tube member, and the second beams of the plurality of sets of beams are angularly offset successively along the longitudinal axis of the tube member.

19. The tube member of claim 18, wherein the first beams of the plurality of sets of beams are angularly offset successively at a constant degree, and the second beams of the plurality of sets of beams are angularly offset successively at a degree equal to the constant degree plus a constant value.

20. The tube member of claim 12, wherein each of the plurality of sets of beams comprises more than two beams.

21. The tube member of claim 12, further comprising a distal portion and a proximal portion positioning the segment of the tube member therebetween, wherein at least one of the distal portion and the proximal portion is provided with a plurality of transverse cuts at a plurality of axial locations along the longitudinal axis of the tube member, the plurality of transverse cuts defining a plurality of rings extending circumferentially around the longitudinal axis and a plurality of sets of beams extending axially along the longitudinal axis to connect the plurality of rings, wherein each of the plurality of sets of beams in the at least one of the distal portion and the proximal portion comprises a first beam and a second beam spaced apart from each other at radial angle of 180 degrees.

22. The tube member of claim 21, wherein the plurality of sets of beams are rotationally offset successively along the longitudinal axis of the tube member.
